(19) United States
(12) Patent Application Publication
(10) Pub. No.: US 2025/0339617 A1
(43) Pub. Date: Nov. 6, 2025

(54) ADAPTIVE UPDATE OF AUTOMATIC INSULIN DELIVERY (AID) CONTROL PARAMETERS

(71) Applicant: INSULET CORPORATION, Acton, MA (US)

(72) Inventors: Joon Bok LEE, Acton, MA (US); Yibin ZHENG, Hartland, WI (US); Jason O'CONNOR, Acton, MA (US)

(21) Appl. No.: 19/270,883

(22) Filed: Jul. 16, 2025

Related U.S. Application Data
(63) Continuation of application No. 17/687,808, filed on Mar. 7, 2022, now Pat. No. 12,370,309.
(60) Provisional application No. 63/158,918, filed on Mar. 10, 2021.
(60) Provisional application No. 63/165,252, filed on Mar. 24, 2021.

(51) Int. Cl. A61M 5/172; A61B 5/00; A61B 5/145; A61M 5/142; G06Q 30/0283; G16H 20/17

(52) U.S. Cl. CPC A61M 5/1723; A61B 5/14532; A61B 5/4839; A61M 5/142; G06Q 30/0283; G16H 20/17; A61M 2005/14208; A61M 2205/52; A61M 2230/201

(57) ABSTRACT

Exemplary embodiments may modify the cost function parameters based on current and projected mean outcomes in blood glucose level control performance. The exemplary embodiments may modify the weight coefficient R for the insulin cost so that the value of R is not fixed and is not based solely on clinical determined values. Exemplary embodiments may also adjust the cost function to address persistent low-level blood glucose level excursions for users. The exemplary embodiments may reduce the penalty of the insulin cost by the sum of the converted insulin cost of the glucose excursions above target for a period divided by a number of cycles of average insulin action time. The AID system reduces the insulin cost by the lack of insulin in previous cycles.

### RELATED APPLICATIONS

This application is a continuation of U.S. patent application Ser. No. 17/687,808, filed Mar. 7, 2022, which claims the benefit of U.S. Provisional Patent Application No. 63/158,918, filed Mar. 10, 2021, and U.S. Provisional Patent Application No. 63/165,252, filed Mar. 24, 2021, the contents of which are incorporated herein by reference in their entirety.

### BACKGROUND

Automatic Insulin Delivery (AID) systems automatically deliver insulin to a user via a delivery mechanism, like an insulin pump. AID systems typically deliver basal insulin to a user, while the user can manually deliver insulin boluses as needed and/or can prompt the insulin pump to deliver insulin boluses as needed. Traditionally, basal insulin accounts for about half of the insulin delivered to a user per day.

AID systems may include a closed control loop that seeks to keep the user's blood glucose level close to a target blood glucose level. In a typical AID system, a controller receives a current blood glucose level reading and compares the current blood glucose level to the target blood glucose level and adjusts the basal insulin delivery to attempt to reduce the difference between the target blood glucose level and the current blood glucose level. In some AID systems, a Model Predictive Control (MPC) approach is adopted. In determining the adjusted basal dosage amount, the controller of the AID system may use a cost function to select the adjusted dosage amount. Specifically, the controller may apply the cost function for a number of possible dosage amounts and select the lowest cost dosage as the adjusted dosage. In other words, the controller seeks to optimize by choosing the lowest cost dosage option.

In one common formulation of the cost function, the cost function is the sum of a weighted glucose cost and a weighted insulin cost. The glucose cost represents the difference between the projected trajectory of the user's blood glucose level over an interval should the adjusted basal amount be chosen for delivery given their current blood glucose level and the target blood glucose level. The glucose cost penalizes positive blood glucose level excursions from the target blood glucose level. The insulin cost represents the difference between the projected insulin trajectory interval over a period should the adjusted basal amount be chosen for delivery and the ideal basal insulin dosage. The insulin cost penalizes insulin excursions above the ideal basal dosage.

The weights of the glucose cost and the insulin cost are determined by weight coefficients Q and R. Q is the weight coefficient for the glucose cost, and R is the weight coefficient for insulin cost. The ratio of Q to R is a key parameter for determining the aggressiveness of adaptation such that blood glucose level excursions will be weighed more heavily than insulin excursions. These weight coefficients Q and R conventionally are fixed based on clinical parameters for the user. Thus, the insulin delivery of the AID system will not vary for a fixed set of clinical parameters for the user. As a result, clinical parameters must change for the AID system to improve the control performance at a given blood glucose level. This is problematic in that the AID system may, as a result, not be performing well and does not adjust the control parameters to perform better.

Given the cost function formulation and weight coefficients, conventional AID systems tend to respond conservatively to persistent but small (“low-level”) blood glucose level excursions that are slightly above the target blood glucose level. These low-level blood glucose level excursions contribute little to the cost function and as a result, have little effect in increasing the insulin delivered to eliminate or reduce such blood glucose level excursions. This conservative response is intentional for multiple reasons. First, blood glucose level readings may be inaccurate, and the AID system does not want to respond too aggressively to such inaccurate readings. Second, the risk of hypoglycemia is viewed as more worrisome than the risk of hyperglycemia, so the response is biased towards being conservative so as to reduce the risk of delivering too much insulin and driving the user into hypoglycemia. Thus, conventional AID systems are biased against over delivery of insulin.

This conservative approach may be problematic. Persistent low magnitude glucose excursions are not desirable. Such excursions may have negative health consequences for users.

### SUMMARY

In accordance with a first inventive aspect, a device for controlling insulin deliveries to a user by an insulin pump includes a glucose sensor interface with a glucose sensor to obtain glucose readings for the user from the glucose sensor and an insulin pump interface for communicating with the insulin pump to control delivery of insulin to the user by the insulin pump. The device further includes a processor configured to implement a control loop to control the delivery of insulin by the insulin pump. The processor selects an insulin delivery dosage for a next delivery among the delivery dosage options that has a best cost function value. The cost function for each of the delivery dosage options has a glucose cost component reflective of a difference between a glucose level that the delivery dosage option is predicted to produce for the user and a projected glucose level with basal insulin delivery. The cost function also has an insulin cost component reflective of a difference between a deviation of the delivery dosage option from a current basal insulin dosage and a converted amount of insulin needed to compensate for glucose excursions above a target for an interval of time. The cost function has a glucose cost weight coefficient for weighting the glucose cost component and has an insulin cost weight coefficient for weighting the insulin cost component.

The device for controlling insulin deliveries may be a drug delivery device that includes the insulin pump. The device for controlling insulin deliveries may be a management device for the insulin pump that does not include the insulin pump. The processor may be configured to calculate the converted amount of insulin needed to compensate for glucose excursions above the target for the interval of time by determining a magnitude of the glucose excursions above the target for the interval. The processor may be configured to determine the magnitude of the glucose excursions above the target for interval by summing glucose excursions above a target of blood glucose level for each cycle in the interval. The processor may be configured for determining the converted amount of insulin needed to compensate for glucose excursions above the target for the interval by applying a conversion factor to the determined magnitude of the glucose excursions above the target.

In accordance with another inventive aspect, a device for controlling insulin deliveries to a user by an insulin pump includes a glucose sensor interface with a glucose sensor to obtain glucose readings for the user from the glucose sensor and an insulin pump interface for communicating with the insulin pump to control delivery of insulin to the user by the insulin pump. The device also includes one or more processors configured to implement a control loop to control the delivery of insulin by the insulin pump such that the processor selects an insulin delivery dosage for a next delivery among the delivery dosage options that has a best cost function value, and the processor also is configured to implement a parallel integral control approach that requests an additional insulin dosage from the insulin pump to eliminate positive glucose excursions that are not eliminated by the control loop.

The one or more processors may be configured so that the parallel integral approach does not request insulin when there are not positive glucose excursions to be eliminated. The device for controlling insulin deliveries may be one of an insulin delivery device or a management device for the for controlling an insulin delivery device. The one or more processors may be configured so that the parallel integral approach determines an amount of insulin needed to eliminate a current magnitude of a positive glucose excursion. The one or more processors may be configured so that the parallel integral approach determines an aggregate magnitude of glucose excursions for a past number of cycles. Further, the one or more processors may be configured so that the parallel integral approach determines a product of the aggregate magnitude of glucose excursions for a past number of cycles and a tuning factor, and the one or more processors may be configured so that the parallel integral approach selects either the amount of insulin needed to eliminate a current magnitude of a positive glucose excursion or the product as the additional insulin dosage.

In accordance with an additional inventive aspect, a device for controlling insulin deliveries to a user by an insulin pump includes a glucose sensor interface with a glucose sensor to obtain glucose readings for the user from the glucose sensor and an insulin pump interface for communicating with the insulin pump to control delivery of insulin to the user by the insulin pump. The device further includes a processor configured to implement a control loop to control the delivery of insulin by the insulin pump. The processor selects an insulin delivery dosage for a next delivery among the delivery dosage options that has a best cost function value. The cost function for each of the delivery dosage options has a glucose cost component reflective of a difference between a glucose level that the delivery dosage option is predicted to produce for the user and a target glucose level for the user. The cost function also has an insulin cost component reflective of a deviation of the delivery dosage option from a current basal insulin dosage. The cost function includes a glucose cost weight coefficient for weighting the glucose cost component and an insulin cost weight coefficient for weighting the insulin cost component. The insulin cost weight coefficient is based on a ratio of time in a desired range for glucose values of the user and a maximum time in the desired range from a history of glucose values for the user.

The insulin cost weight coefficient may also be based on a base value for the insulin cost weight coefficient. The maximum time in the desired range may be a percentage value. The processor may be configured to determine the maximum time in the desired range based on an average target blood glucose value of the user over the history of glucose values for the user. The processor may be configured to determine the maximum time in the desired range based additionally on a percentage of time that glucose values for the user were in range over the history of glucose values for the user. The device for controlling insulin deliveries may be one of an insulin delivery device or a management device for an insulin delivery device. The insulin cost weight coefficient may increase in value as the ratio of time in a desired range for glucose values of the user and maximum time in the desired range from a history of glucose values for the user increases.

### BRIEF DESCRIPTION OF THE DRAWINGS

FIG. 1 depicts an illustrative drug delivery system that is suitable for delivering insulin to a user in accordance with exemplary embodiments.

FIG. 2 illustrates a simplified block diagram of an example of a control loop suitable for exemplary embodiments.

FIG. 3 depicts a flowchart of illustrative steps that may be performed by exemplary embodiments in determining what dose of insulin to deliver the user as part of the closed loop control system.

FIG. 4 depicts a flowchart of illustrative steps that may be performed to determine the cost of an insulin dosage with an adjusted cost function in exemplary embodiments.

FIG. 5 depicts a flowchart of illustrative steps that may be performed when a parallel controller is provided to reduce low-level glucose excursions.

FIG. 6 depicts a flowchart of illustrative steps that may be performed to calculate Iₒᵤₜ in exemplary embodiments.

FIG. 7 depicts a flowchart of illustrative steps that may be performed in exemplary embodiments to determine the weight coefficient R.

FIG. 8 depicts a flowchart of illustrative steps that may be performed to determine TIRₘₐₓ in exemplary embodiments.

### DETAILED DESCRIPTION

Exemplary embodiments may address the above-described problems of conventional AID systems. Exemplary embodiments may modify the cost function parameters based on current and projected mean outcomes in blood glucose level control performance. For instance, the exemplary embodiments may modify the weight coefficient R for the insulin cost so that the value of R is not fixed and is not based solely on clinically determined values. The modification may be bounded by the known impacts of the maximum impact of modifying the user's personal therapy parameters. This allows for more customized and better control of blood glucose levels for users. Specifically, if it is determined that the AID system is controlling the blood glucose level of the user well, the cost function is adjusted to be less aggressive (i.e., adapts more slowly), whereas if it is determined that the AID system is controlling the blood glucose level of the user poorly, the cost function is adjusted to be more aggressive (i.e., adapts more quickly). The AID system looks at recent blood glucose level outcomes for the user and the best-case outcome to determine how the AID system is performing in blood glucose level control.

Exemplary embodiments may also adjust the cost function to address persistent low-level blood glucose level excursions for users. The exemplary embodiments may reduce the penalty of the insulin cost by the sum of the converted insulin cost of the glucose excursions above target for a period divided by a number of cycles of average insulin action time. The AID system reduces the insulin cost by the lack of insulin in previous cycles. As a result, the persistent low-level glucose excursions are more likely to be addressed by the AID system.

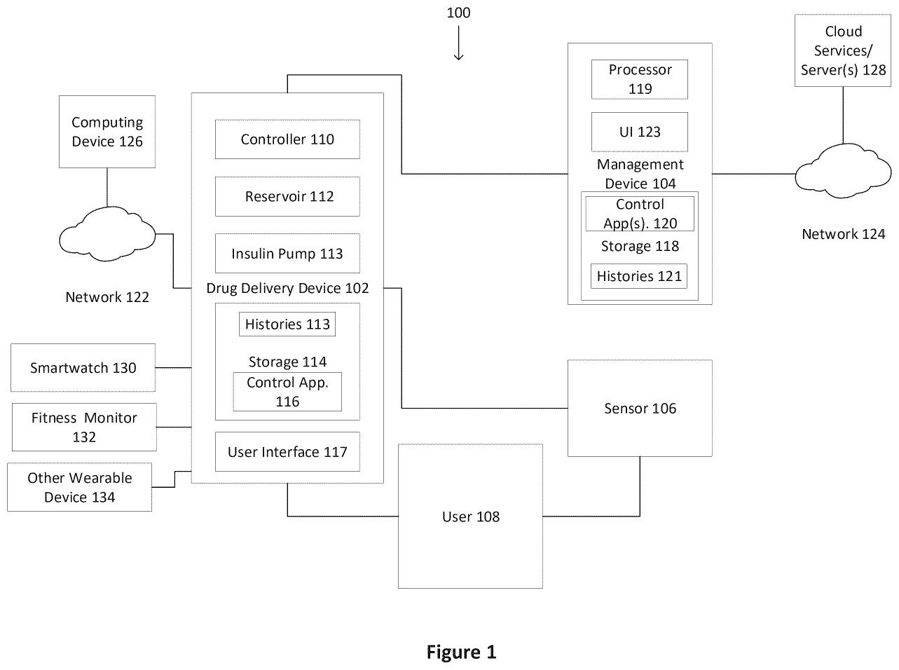

FIG. 1 depicts an illustrative drug delivery system 100 that is suitable for delivering insulin to a user 108 in accordance with exemplary embodiments. The drug delivery system 100 includes a drug delivery device 102. The drug delivery device 102 may be a wearable device that is worn on the body of the user 108. The drug delivery device 102 may be directly coupled to a user (e.g., directly attached to a body part and/or skin of the user 108 via an adhesive or the like). In an example, a surface of the drug delivery device 102 may include an adhesive to facilitate attachment to the user 108.

The drug delivery device 102 may include a controller 110. The controller 110 may be implemented in hardware, software, or any combination thereof. The controller 110 may, for example, be a microprocessor, a logic circuit, a field programmable gate array (FPGA), an application specific integrated circuit (ASIC) or a microcontroller coupled to a memory. The controller 110 may maintain a date and time as well as other functions (e.g., calculations or the like). The controller 110 may be operable to execute a control application 116 stored in the storage 114 that enables the controller 110 to direct operation of the drug delivery device 102. The control application 116 may control insulin delivery to the user 108 per an AID control approach as describe herein. The storage 114 may hold histories 113 for a user, such as a history of automated insulin deliveries, a history of bolus insulin deliveries, meal event history, exercise event history and the like. In addition, the controller 110 may be operable to receive data or information. The storage 114 may include both primary memory and secondary memory. The storage may include random access memory (RAM), read only memory (ROM), optical storage, magnetic storage, removable storage media, solid state storage or the like.

The drug delivery device 102 may include a reservoir 112 for storing insulin for delivery to the user 108 as warranted. A fluid path to the user 108 may be provided, and the drug delivery device 102 may expel the insulin from the reservoir 112 to deliver the insulin to the user 108 via the fluid path. The fluid path may, for example, include tubing coupling the drug delivery device 102 to the user 108 (e.g., tubing coupling a cannula to the reservoir 112).

There may be one or more communications links with one or more devices physically separated from the drug delivery device 102 including, for example, a management device 104 of the user and/or a caregiver of the user and/or a sensor 106. The communication links may include any wired or wireless communication link operating according to any known communications protocol or standard, such as Bluetooth®, Wi-Fi, a near-field communication standard, a cellular standard, or any other wireless protocol The drug delivery device 102 may also include a user interface 117, such as an integrated display device for displaying information to the user 108 and in some embodiments, receiving information from the user 108. The user interface 117 may include a touchscreen and/or one or more input devices, such as buttons, knob or a keyboard.

The drug delivery device 102 may interface with a network 122. The network 122 may include a local area network (LAN), a wide area network (WAN) or a combination therein. A computing device 126 may be interfaced with the network, and the computing device may communicate with the insulin delivery device 102.

The drug delivery system 100 may include a sensor 106 for sensing the levels of one or more analytes. The sensor 106 may be coupled to the user 108 by, for example, adhesive or the like and may provide information or data on one or more medical conditions and/or physical attributes of the user 108. The sensor 106 may, in some exemplary embodiments provide periodic blood glucose concentration measurements and may be a continuous glucose monitor (CGM), or another type of device or sensor that provides blood glucose measurements. The sensor 106 may be physically separate from the drug delivery device 102 or may be an integrated component thereof. The sensor 106 may provide the controller 110 with data indicative of measured or detected blood glucose levels of the user 108. The information or data provided by the sensor 106 may be used to adjust drug delivery operations of the drug delivery device 102.

The drug delivery system 100 may also include the management device 104. In some embodiments, no management device is needed. The management device 104 may be a special purpose device, such as a dedicated personal diabetes manager (PDM) device. The management device 104 may be a programmed general-purpose device, such as any portable electronic device including, for example, a dedicated controller, such as processor, a micro-controller or the like. The management device 104 may be used to program or adjust operation of the drug delivery device 102 and/or the sensor 104. The management device 104 may be any portable electronic device including, for example, a dedicated device, a smartphone, a smartwatch or a tablet. In the depicted example, the management device 104 may include a processor 119 and a storage 118. The processor 119 may execute processes to manage a user's blood glucose levels and for control the delivery of the drug or therapeutic agent to the user 108. The processor 119 may also be operable to execute programming code stored in the storage 118. For example, the storage may be operable to store one or more control applications 120 for execution by the processor 119. The one or more control applications 120 may be responsible for controlling the drug delivery device 102, including the AID delivery of insulin to the user 108. The storage 118 may store the one or more control applications 120, histories 121 like those described above for the insulin delivery device 102 and other data and/or programs.

The management device 104 may include a user interface (UI) 123 for communicating with the user 108. The user interface 123 may include a display, such as a touchscreen, for displaying information. The touchscreen may also be used to receive input when it is a touch screen. The user interface 123 may also include input elements, such as a keyboard, button, knob or the like.

The management device 104 may interface with a network 124, such as a LAN or WAN or combination of such networks. The management device 104 may communicate over network 124 with one or more servers or cloud services 128.

Other devices, like smartwatch 130, fitness monitor 132 and wearable device 134 may be part of the drug delivery system 100. These devices may communicate with the drug delivery device 102 to receive information and/or issue commands to the drug delivery device 102. These devices 130, 132 and 134 may execute computer programming instructions to perform some of the control functions otherwise performed by controller 110 or processor 119. These devices 130, 132 and 134 may include displays for displaying information such as current blood glucose level, insulin on board, insulin deliver history, etc. The display may show a user interface for providing input, such as request a change in basal insulin dosage or delivery of a bolus of insulin. These devices 130, 132 and 134 may also have wireless communication connections with the sensor 106 to directly receive blood glucose level data.

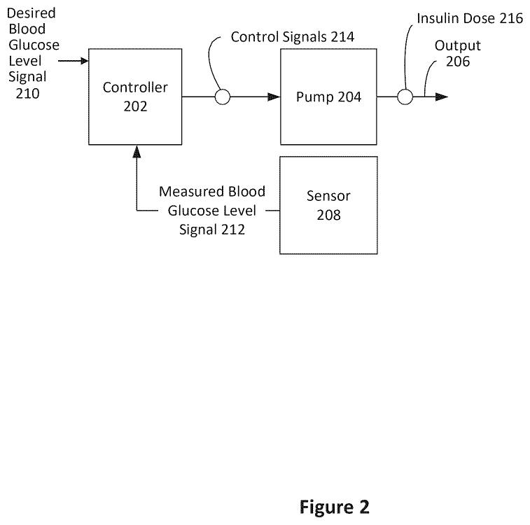

As was mentioned above, a control loop may be provided to adjust the basal delivery dosage based on current blood glucose level readings. FIG. 2 illustrates a simplified block diagram of an example of such a control loop 200 suitable for practicing an exemplary embodiment. The example control loop 200 may include a controller 202, a pump mechanism or other fluid extraction mechanism 204 (hereinafter “pump 204”), and a sensor 208. The controller 202, pump 204, and sensor 208 may be communicatively coupled to one another via a wired or wireless communication paths. The sensor 208 may be a glucose monitor such as, for example, a continuous glucose monitor (CGM) 208. The CGM 208 may, for example, be operable to measure blood glucose values of a user to generate the measured actual blood glucose level signal 212.

As shown in the example, the controller 202 may receive a desired blood glucose level signal 210, which may be a first signal, indicating a desired blood glucose level or range for a user. The desired blood glucose level signal 210 may be received from a user interface to the controller or other device, or by an algorithm that automatically determines a desired blood glucose level for a user. The sensor 208 may be coupled to the user and be operable to measure an approximate value of an actual blood glucose level of the user. The measured blood glucose value, the actual blood glucose level, the approximate measured value of the actual blood glucose level are only approximate values of a user's blood glucose level, and it should be understood that there may be errors in the measured blood glucose levels. The errors may, for example, be attributable to a number of factors such as age of the sensor 208, location of the sensor 208 on a body of a user, environmental factors (e.g., altitude, humidity, barometric pressure), or the like. The terms measured blood glucose value, actual blood glucose level, approximate measured value of the actual blood glucose level may be used interchangeably throughout the specification and drawings. In response to the measured blood glucose level or value, the sensor 208 generate a signal indicating the measured blood glucose value. As shown in the example, the controller 202 may also receive from the sensor 208 via a communication path, a measured blood glucose level signal 212, which may be a second signal, indicating an approximate measured value of the actual blood glucose level of the user.

Based on the desired blood glucose level signal 210 and the measured actual blood glucose level signal 212, the controller 202 may generate one or more control signals 214 for directing operation of the pump 204. For example, one of the control signals 214 may cause the pump 204 to deliver a dose of insulin 216 to a user via output 206. The dose of insulin 216 may, for example, be determined based on a difference between the desired blood glucose level signal 210 and the actual blood glucose signal level 212. The cost function referenced above plays a role in determining the dosage as part of the closed loop control system as will be described below. The dose of insulin 216 may be determined as an appropriate amount of insulin to drive the actual blood glucose level of the user to the desired blood glucose level. Based on operation of the pump 204 as determined by the control signals 214, the user may receive the insulin 216 from the pump 204.

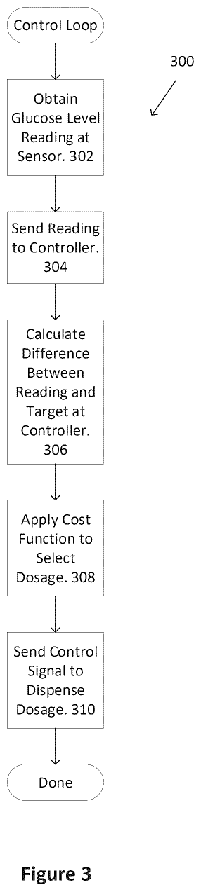

FIG. 3 depicts a flowchart 300 of steps that may be performed by exemplary embodiments of the AID system in determining what dose of insulin to deliver to the user as part of the closed loop control system. These steps may be performed by controller 110, processor 119 or other components (at least in part), like smartwatch 130, fitness monitor or wearable device 134. That said, for purposes of simplicity below, we will just refer to controller 110. Initially, as was described above relative to FIG. 2, a blood glucose level reading is obtained by the sensor 208 (302). The blood glucose level reading is sent via a signal 212 to the controller 202 (304). The controller 202 calculates an error value as the difference between the measured blood glucose level 212 and the desired BG level 210 (306). The closed loop control system attempts to minimize the aggregate penalty of the cost function over a wide range of possible dosages. The cost function is applied to the possible dosages, and the dosage with the best cost function value is selected (308). Depending on how the cost function is configured, the best value may be the lowest value or the highest value. The cost function used in exemplary embodiments will be described in more below. A control signal 214 may be generated by the controller 202 and sent to the pump 204 to cause the pump to deliver the desired insulin dose 216 to the user (310).

As discussed above, the exemplary embodiments may adjust the cost function to address persistent low-level blood glucose level excursions for users. As a starting point, it is helpful to review a typical conventional cost function. A typical formulation for cost J is:

$$J = Q \cdot {\sum }_{i = 1}^{M} {{G}_{p} ( i )}^{2} + R \cdot {\sum }_{i = 1}^{n} {{I}_{p} ( i )}^{2}$$

where Q and R are weight coefficients as mentioned above, Gₚ(i)² is the square of the deviation between the projected blood glucose level for an insulin dosage at cycle i and the projected blood glucose level for the basal insulin dosage, M is the number of cycles in the prediction horizon, Iₚ(i)² is the square of the deviation between the projected insulin delivered at cycle i and the insulin for basal insulin delivery, and n is the control horizon in cycles. Thus,

$$Q \cdot {\sum }_{i = 1}^{M} {{G}_{p} ( i )}^{2}$$

is the weighted glucose cost, and

$$R \cdot {\sum }_{i = 1}^{n} {{I}_{p} ( i )}^{2}$$

is the weighted insulin cost. The total cost J is the sum of the weighted glucose cost and the weighted insulin cost. A cycle has a fixed interval, such 5 minutes.

The exemplary embodiments may modify the cost function to increasingly penalize blood glucose level excursions from the target blood glucose level by increasing basal insulin delivery over time. This may be done by reducing the insulin cost component in the cost function. Specifically, an Iₒᵤₜ variable may be introduced into the cost function formula to account for the additional insulin needed to reduce the low-level blood glucose level excursions. This additional insulin may be subtracted from insulin cost.

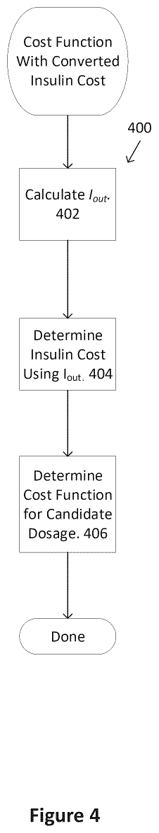

FIG. 4 depicts a flowchart 400 of illustrative steps that may be performed to determine the cost of an insulin dosage with an adjusted cost function. The Iₒᵤₜ variable is calculated over the past h cycles. The cycles may be fixed length, such as 5 minutes per cycle. A suitable formula for calculating Iₒᵤₜ in an exemplary embodiment is:

$${I}_{out} = {K}_{i} {\sum }_{i = 1}^{h} \frac{{G}_{h} ( t - i ) - SP ( t - i )}{\frac{CFrule}{TDI} \cdot τ}$$

Where Gₕ(t−i) is the blood glucose level at the ith cycle before cycle t, SP(t−i) is the target blood glucose level at the ith cycle before cycle t, CFrule is the correction factor for the user indicating how much 1 unit of insulin will lower the blood glucose level of the user over a period of time (like 2 to 4 hours), TDI is the total daily insulin for the user, τ is a parameter relating to the peak insulin action time for the user and Kᵢ is a tuning factor. Suitable example values for some of the variables are h to be 6 (i.e., 30 minutes), CFrule to be 1800 and τ to be 18 (i.e., 90 minutes or 18 5-minute cycles) with a range of 6 to 36.

The formula aggregates the blood glucose level excursions (see the numerator) over the last h cycles via the summation and determines the additional insulin requirements (see the conversion into insulin requirements in the denominator) required to bring the excursions to the target blood glucose level.

Referring again to FIG. 4, with Iₒᵤₜ calculated, the insulin cost for a proposed insulin dose being considered by the controller 110 as part of the AID approach may be determined at 404. A suitable cost function formula that is adaptive for cost J_{new} is:

$${J}_{new} = Q \cdot {\sum }_{i = 1}^{M} {{G}_{p} ( i )}^{2} + R \cdot {\sum }_{i = 1}^{n} {( {I}_{p} ( i ) - {I}_{out} )}^{2} .$$

As can be seen, the difference in this cost function relative to the conventional cost function is that the insulin cost is calculated differently. The insulin cost subtracts out Iₒᵤₜ. Thus, there is less of a penalty for additional insulin, and the basal dosage amount may increase to address the persistent low-level blood glucose level excursions.

With the insulin cost calculated as such, the weighted glucose cost may be determined and the cost function for the candidate dosage determined at 406.

In the above-described approach of exemplary embodiments, the controller 110 makes an adjustment to the insulin cost in the cost function to eliminate the low-level blood glucose level excursions as part of an AID control approach. Alternatively, the low-level blood glucose level excursions may be addressed by a separate mechanism that runs in conjunction with the un-modified AID control approach. A parallel controller may perform the operations described below to address the persistent low-level blood glucose level excursions.

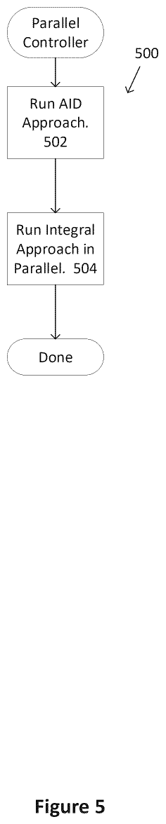

FIG. 5 depicts a flowchart 500 of illustrative steps for such exemplary embodiments. The AID approach is run as described above for conventional systems using a conventional cost function 502. The integral approach (labelled as such because it determines the integral of the insulin needed to reduce or eliminate the blood glucose level excursions) described below is run in parallel on a parallel controller 504. The parallel controller may be part of a single controller that performs the two control approaches in parallel or may run on separate controllers on a same drug delivery device or management device.

The controller running in parallel may make a request for an insulin dosage that is separate from that of the AID control approach of the other controller. The aim of the separate request is to provide additional insulin to eliminate or reduce the blood glucose level excursions. The parallel controller may determine the insulin amount requested I_{integral}(t) as:

$${I}_{integral} ( t ) = max ( min ( \frac{{G}_{h} ( t ) - SP ( t )}{\frac{CFrule}{TDI}} , {K}_{i} {\sum }_{i = 1}^{h} ( {G}_{h} ( t - i ) - SP ( t - i ) ) ) , 0 ) .$$

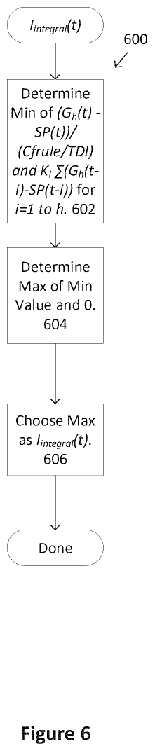

As shown in the flowchart of FIG. 6, the formula calculates the minimum of

$$\frac{{G}_{h} ( t ) - SP ( t )}{\frac{CFrule}{TDI}} and {K}_{i} {\sum }_{i = 1}^{h} ( {G}_{h} ( t - i ) - SP ( t - i ) )$$

at 602. The value

$$\frac{{G}_{h} ( t ) - SP ( t )}{\frac{CFrule}{TDI}}$$

is the amount of insulin needed to compensate for the excess blood glucose level at time t. The value

$${K}_{i} {\sum }_{i = 1}^{h} ( {G}_{h} ( t - i ) - SP ( t - i ) )$$

is the cumulative difference in the blood glucose level relative to the target blood glucose level (i.e., the total glucose blood level excursions) over the past h cycles multiplied by a tuning factor Kᵢ. In some embodiments, Kᵢ may be made dependent on the user's average insulin requirements, such as TDI. The insulin amount requested is bounded at 0, so the formula takes the maximum or the calculated values and zero. This ensures that the amount request is greater than or equal to zero at 604. The largest value is chosen as I_{integral}(t) at 606.

In exemplary embodiments, the cost function may also be adjusted to modify the aggressiveness of the AID control approach based on an average time a user maintains his or her blood glucose level in an acceptable range. As was mentioned above, the current performance is measured relative to best performance to determine how aggressive to be in adapting the AID control approach. One way of achieving this adjustment in aggressiveness is to modify the R coefficient for insulin cost in the cost function. In exemplary embodiments, the cost function detailed above may be used. Thus, the cost function may be:

$$J = Q \cdot {\sum }_{i = 1}^{M} {{G}_{p} ( i )}^{2} + R \cdot {\sum }_{i = 1}^{n} {{I}_{p} ( i )}^{2}$$

With Q fixed at 1, so that R controls the aggressiveness of the adaptability of the AID control approach.

In some exemplary embodiments, R may be determined by the formula:

$$R = ( 1 - 0.2 \cdot \frac{h}{288} ) \cdot {R}_{base} + 0.2 \cdot {R}_{base} \cdot {( \frac{{TIR}_{h , 70 < CGM < 180}}{{TIR}_{Max}} )}^{2}$$

Where R_{base} is a baseline tuning factor relating to the aggressiveness of the AID control approach, TIR_{h,70<CGM<180} is the percentage of time that the blood glucose level was in the acceptable range between 70 and 180 mg/dL during the h cycles for which there is available blood glucose level history to the controller 110, 288 is the number of cycles per day with 5 minute cycles, and TIR_{Max} is the maximum percentage of time in range for the user over all of the available blood glucose level history.

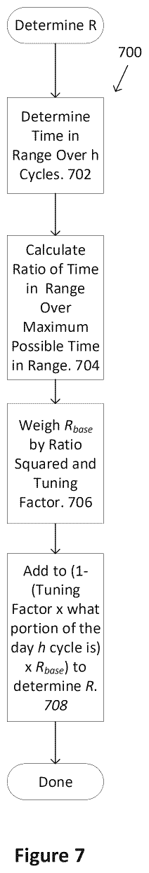

FIG. 7 depicts a flowchart 700 of steps that may be performed in exemplary embodiments to determine R. The percentage of time in range over the h cycles of available blood glucose level history is determined at 702. The percentage of time in range is represented in the above formula for R by the variable TIR_{h,70<CGM<180}. It should be appreciated that other acceptable ranges that differ from the range between 70 and 180 mg/dL may be used in some embodiments. TIR_{Max} is determined as will be detailed below and the ratio

$$\frac{{TIR}_{h , 70 < CGM < 180}}{{TIR}_{Max}}$$

is determined at 704. This ratio represents the amount of time the blood glucose level of the user was in range versus a maximum available time in range. Hence, the ratio is a good estimate of how well the user manages his/her blood glucose level. A large ratio closer to 1 indicates that the user manages his/her blood glucose level well, where a small ratio indicates that the user does not manage his/her blood glucose level well. R_{base} is weighted by the product of the ratio squared times a tuning factor (e.g., 0.2) at 706. Hence, R is larger and the adaptivity is more aggressive when the ratio is higher, and R is smaller and the adaptivity is less aggressive when the ratio is smaller. At 708, the weighted squared ratio is added to

$$( 1 - 0.2 \cdot \frac{h}{288} ) \cdot {R}_{base} .$$

The variable h is the number of cycles of available blood glucose level history for the user. So, if there is more history, the ratio

$$\frac{h}{288}$$

approaches one and the weighted squared ratio contributes more to R. This is because the confidence in the data grows as there is more data available.

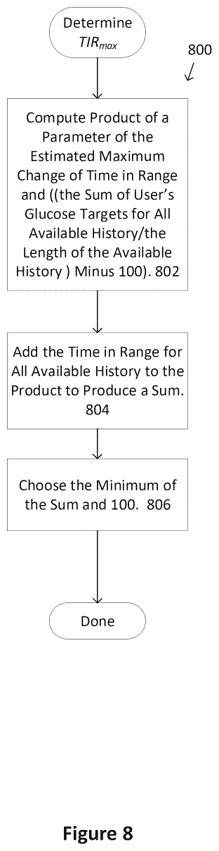

FIG. 8 depicts a flowchart 800 of illustrative steps that may be performed to determine TIRₘₐₓ. A formula for TIRₘₐₓ that may be used in exemplary embodiments is:

$${TIR}_{Max} = min ( 100 , {TIR}_{all history , 70 < CGM < 180} + 0.3 \cdot ( \frac{{\sum }_{i = 1}^{{L}_{all history}} SP ( i )}{{L}_{all history}} - 100 ) )$$

where TIR_{all history,70<CGM<180} is the percentage of time that the blood glucose level of the user was in the acceptable range over all of the available blood glucose level history for the user and L_{all history} is the length of the all available blood glucose level history for the user.

The fraction

$$\frac{{\sum }_{i = 1}^{{L}_{all history}} SP ( i )}{{L}_{all history}}$$

determines the user's average blood glucose level target over the available history. The value 100 is subtracted from this fraction and multiplied by a tuning factor (e.g., 0.3) at 802. The resulting value of the calculation made at 802 is added to the time in range over all of the available blood glucose level history at 804. The smaller of the sum of the calculation made at 804 and 100 is chosen as the maximum percentage in range at 806.

While the discussion has focused on exemplary embodiments herein, it should be appreciated that various changes in form and detail without departing from the intended scope as defined in the appended claims.

1. A drug delivery system, comprising:
a drug delivery device;
one or more processors configured to implement a control loop to control delivery of insulin by the drug delivery device, wherein the one or more processors select an insulin delivery dosage for a next basal insulin dosage delivery among delivery dosage options that has a best value of a cost function and wherein the cost function for each of the delivery dosage options:
has a glucose cost component reflective of a difference between a glucose level that the delivery dosage option is predicted to produce for the user and a glucose level for the user relative to a projected glucose level with basal drug delivery,
has an insulin cost component reflective of a difference between a deviation of the delivery dosage option from a current basal insulin dosage and a converted amount of insulin needed to compensate for glucose excursions above a target for an interval of time,
has a glucose cost weight coefficient for weighting the glucose cost component, and
has an insulin cost weight coefficient for weighting the insulin cost component.

2. The drug delivery system of claim 1, wherein the drug delivery device includes an insulin pump.

3. The drug delivery system of claim 1, wherein the drug delivery system includes a management device for managing the drug delivery device.

4. The drug delivery system of claim 3, wherein the one or more processors are part of the management device.

5. The drug delivery system of claim 1, wherein the interval includes cycles and wherein the one or more processors are configured to determine the magnitude of the glucose excursions above the target for the interval of time by summing glucose excursions above a target of blood glucose level for each of the cycles in the interval.

6. The drug delivery system of claim 5, wherein the one or more processors are configured for determining the converted amount of insulin needed to compensate for glucose excursions above the target for the interval of time by applying a conversion factor to the determined magnitude of the glucose excursions above the target.

7. A drug delivery system, comprising:
an insulin pump;
one or more processors configured to:
implement a control loop to control delivery of insulin by the insulin pump such that the one or more processors select an insulin delivery dosage for a next delivery among delivery dosage options that has a best value of a cost function, and
implement a parallel integral control approach that requests an additional insulin dosage from the insulin pump to eliminate positive glucose excursions that are not eliminated by the control loop.

8. The drug delivery system of claim 7, wherein the one or more processors are either part of the insulin pump or part of a management device for managing the insulin pump.

9. The drug delivery system of claim 8, wherein the management device is a dedicated device or a smartphone.

10. The drug delivery system of claim 8, wherein the one or more processors are configured so that the parallel integral approach determines an amount of insulin needed to eliminate a current magnitude of a positive glucose excursion.

11. The drug delivery system of claim 10, wherein the one or more processors are configured so that the parallel integral approach determines an aggregate magnitude of glucose excursions for a past number of cycles.

12. The drug delivery system of claim 11, wherein the one or more processors are configured so that the parallel integral approach determines a product of the aggregate magnitude of glucose excursions for a past number of cycles and a tuning factor.

13. The drug delivery system of claim 12, wherein the one or more processors are configured so that the parallel integral approach selects either the amount of insulin needed to eliminate a current magnitude of a positive glucose excursion or the product as the additional insulin dosage.

14. A drug delivery system, comprising:
an insulin pump;
one or more processors configured to implement a control loop to control delivery of insulin by the insulin pump, wherein the one or more processors select an insulin delivery dosage for a next delivery among the delivery dosage options that has a best value of a cost function and wherein the cost function for each of the delivery dosage options:
has a glucose cost component reflective of a difference between a glucose level that the delivery dosage option is predicted to produce for the user and a target glucose level for the user,
has an insulin cost component reflective of a deviation of the delivery dosage option from a current basal insulin dosage,
has a glucose cost weight coefficient for weighting the glucose cost component, and
has an insulin cost weight coefficient for weighting the insulin cost component, wherein the insulin cost weight coefficient is based on a ratio of time in a desired range for glucose values of the user and maximum time in the desired range from a history of glucose values for the user.

15. The drug delivery system of claim 14, wherein the insulin cost weight coefficient is also based on a base value for the insulin cost weight coefficient.

16. The drug delivery system of claim 14, wherein the maximum time in the desired range is a percentage value.

17. The drug delivery system of claim 14, wherein the one or more processors are configured to determine the maximum time in the desired range based on an average target blood glucose value of the user over the history of glucose values for the user.

18. The drug delivery system of claim 17, wherein the one or more processors are configured to determine the maximum time in the desired range based additionally on a percentage of time that glucose values for the user were in range over the history of glucose values for the user.

19. The drug delivery system of claim 14, wherein the one or more processors are part of the insulin pump or part of a management device for the insulin pump.

20. The drug delivery system of claim 14, further comprising a non-transitory storage medium for storing the history of glucose values for the user.
